(19) United States
(12) Patent Application Publication
(10) Pub. No.: US 2013/0236432 A1
(43) Pub. Date: Sep. 12, 2013

(54) METHOD AND DEVICE FOR MULTIPLYING AND DIFFERENTIATING CELLS IN THE PRESENCE OF GROWTH FACTORS AND OF A BIOLOGICAL MATRIX OR OF A SUPPORTING STRUCTURE

(71) Applicant: Augustinus BADER, Klinga (DE)

(72) Inventor: Augustinus BADER, Klinga (DE)

(73) Assignee: Augustinus BADER, Klinga (DE)

(21) Appl. No.: 13/867,842

(22) Filed: Apr. 22, 2013

Related U.S. Application Data
(63) Continuation of application No. 10/518,628, filed on Jun. 27, 2005, now abandoned.

(30) Foreign Application Priority Data
Jun. 20, 2002 (DE) 10227611.0
Jul. 26, 2002 (DE) 10234204.0

(51) Int. Cl. A61K 38/18; A61K 35/12

(52) U.S. Cl. CPC A61K 38/1816; A61K 35/12
USPC 424/93.7; 514/7.7

(57) ABSTRACT

The invention relates to an in-vitro and in-vivo method for multiplying and differentiating cells, during which the growth process of the cells is initiated or terminated and structurally directed by the use of growth factors thrombopoietin (TPO) and/or erythropoietin (EPO), and/or growth hormone (GH), particularly human growth hormone (HGH), and/or somatostatin and/or leukemia inhibitory factor (LIF) and/or ciliary neurotropic factor (CNTF). The invention also relates to a biological matrix or supporting structure containing the aforementioned growth factors, and to a method and device for the production thereof and for carrying out the inventive method.

The present invention relates to the use of at least one growth factor in isolated form for the cultivation of primarily differentiated cells, for the locally specific and/or directed differentiation of adult cells and/or for the regeneration of bones, tissues and/or endocrine organs.

In ontogenesis, that is to say the development of the individual organism, there is expression of growth factors which are able to initiate fundamental structural processes which are numerical in relation to the number of cells. In the growing organism, the ability for structural repairs through regeneration is increasingly lost because these growth factors are no longer expressed. Factors of the bone marrow and of the blood-forming organs are coupled in time to growth processes of other organs during specific ontogenetic processes.

One disadvantage of known growth factors such as, for example, epidermal growth factor (EGF), vascular endothelial growth factor (VEGF) or hepatocyte growth factor (HGF) is that the multiplication processes, especially on use of primary cells in vitro, are limited and that the use in vivo is problematic because of possible side effects such as, for example, the activation of oncogenes.

It has to date been assumed that tissue extracts such as, for example, from the pituitary or the hypothalamus are particularly suitable for bringing about multiplication of hepatocyte cells (see, for example, U.S. Pat. No. 6,008,047). Such animal or, occasionally, human extracts have already been added to cell cultures. The use of animal or human tissue extracts is, however, problematic in laboratory work or in clinical use owing to transmissible viral diseases such as, for example, BSE, pig or sheep viruses. The use of such extracts demonstrates the lack of knowledge about the actually relevant factors and their potential uses and effects. A further substantial disadvantage is that through such heterogeneous extracts, which are generally difficult to define and depend considerably on the source used, there is also introduction into the culture of factors which, in some circumstances, bring about unwanted side effects or properties on clinical use. Accurate knowledge of the factors and controlled dosage thereof would therefore be both an important factor for being able to multiply and differentiate cells, especially in the area of tissue engineering, appropriately, and for inducing structural processes of three-dimensional (3-D) regeneration.

Such structural tasks are a priority in particular for tissue engineering, although 3-D growth and its initiation is not as yet understood. Although conventional approaches such as aggregate cultures achieve a high density, they must be built up with cells which have been preexpanded or isolated from primary tissues, e.g. hepatocytes. An inductive growth process into a defined, predetermined structure has not to date been possible.

On the contrary, cells such as, for example, hepatocytes are still embedded after the multiplication phases in a gel in order to avoid the formation of further, also large, aggregate (superaggregates). These gels are two-dimensional in extent, comprise a high cell density and therefore stop cell multiplication. Such 2-D gel inclusions, which result in layers, have already been described by Bader et al. (1995), Artif. Organs, 19, 368-374, as sandwich model or gel entrapment. Although the embedding of aggregates in gels results in an improvement in maintenance of differentiation, it does not result in further growth.

A shape-creating growth from a few precursor cells to a 3-D structure and an inductive behavior for neighborhood processes in the sense of tissue regeneration in vitro and in vivo has not to date been possible. However, inductive growth behavior of cells means a considerable innovation in particular for therapeutic or biotechnological processes. Such a growth behavior should, assisted by a 3-D supporting matrix, allow growth not only in the sense of colonization or structural remodeling but in fact be able to allow directed de novo formation from an induction nucleus. Such processes take place in ontogenesis and build upon a pre-existing anlage.

It is known merely that growth factors, especially in the case of neuronal progenitors of fetal origin such as, for example, leukemia inhibitory factor (LIF), ciliary neurotropic factor (CNTF), glial derived neurotrophic factor (GDNF) or nerve growth factor (NGF), make a proliferation phase of undifferentiated neurons possible. However, after differentiation is achieved, these factors are no longer able to act.

In tissue engineering there is in addition the problem that patient-specific adult cell systems which are already differentiated further than fetal cells are used. In addition, coculture situations apply in situ and in vitro but are not taken into account in conventional usage. On the contrary, attempts are even made for example to avoid cocultures of endothelial cells, macrophages and fibroblasts, as occur in the liver, on expansion of the parenchymal liver cells, because they are unwanted. However, it is now known that the presence of these so-called non-parenchymal cells in differentiated cultures make a substantial contribution to the differentiation.

It is therefore desirable to provide a multiplication method in vitro and/or a regeneration method in vivo which is able substantially to maintain the physiological state of the cell systems and make substantially structural growth possible.

It has now been found that the use of the growth factors thrombopoietin (TPO) and/or erythropoietin (EPO) and/or growth hormone (GH), and/or somatostatin and/or leukemia inhibitory factor (LIF) and/or ciliary neurotropic factor (CNTF) initiates and terminates, and structurally guides, the multiplication and differentiation of cells.

Surprisingly, this has brought about not only a multiplication of cells but also an induction of structural processes, in particular a locally specific cell multiplication and directed differentiation is brought about by inductive effect on an implant in place (in situ) for example via a so-called homing process. This means that the growth hormones are able to induce but also terminate these structural processes.

The invention therefore relates to a method for multiplying and differentiating cells in vitro, in which the growth process of the cells is initiated and terminated, and structurally guided, by the use of the growth factors TPO and/or EPO and/or GH, especially HGH and/or somatostatin and/or LIF and/or CNTF.

Thus, TPO is also known for example as c-Mpl ligand, mpl ligand, megapoietin or megakariocyte growth and development factor and has to date not been employed in the culturing of, for example, adult hepatocytes or other primary cells apart from platelets and their precursors. TPO is essentially necessary for the development and proliferation of megakariocytes and platelets and thus for the formation of blood platelets. TPO is constitutively produced in the liver and in the kidneys as 332 amino acid-long protein.

Additional growth factors which can be employed according to the present invention are transforming growth factor beta (TGF beta), prostaglandins, granulocyte-macrophage stimulating faster (GM-CSF), growth hormone releasing hormone (GHRH), thyrotropin-releasing hormone (TRH), gonadotropin-releasing hormone (GnRH), corticotropin-releasing hormone (CRH), dopamine, antidiuretic hormone (ADH), oxytocin, prolactin, adrenocorticotropin, beta-celltropin, lutrotropin and/or vasopressin.

Besides cessation or reduction of the supply of the described growth factors to the culture, somatostatin and/or TGF beta and/or prostaglandins are also suitable for terminating the growth process of the invention.

The individual concentrations of the growth factors in solution are normally about 1 to about 100 ng/ml, preferably about 10 to about 50 ng/ml, in particular about 10 to about 20 ng/ml. However, in the case of local coatings, the concentrations of the growth factors may also be a multiple thereof.

For example, in the case of regeneration of endocrine organs, interaction of the growth factors, in particular of growth hormone releasing hormone (GHRH), thyrotropin-releasing hormone (TRH), gonadotropin-releasing hormone (GnRH), corticotrophin-releasing hormone (CRH), somatostatin, dopamines, antidiuretic hormone (ADH) and/or oxytocin, with the supporting matrix may induce endocrine differentiation and/or growth in situ.

It is additionally possible to employ prolactin, adrenocorticotropin, beta-celltropin, lutrotropin and/or vasopressin for the structural processes.

In a further embodiment it is additionally possible to employ one or more nerve regeneration factors, preferably nerve growth factor (NGF) and/or one or more vessel regeneration factors, preferably vascular endothelial growth factor (VEGF) and/or platelet derived growth factor (PDGF).

In the presence of endothelial cells it is possible to achieve an endothelialization of the cells and thus an optimal hemocompatibility.

Said growth factors can generally be purchased commercially but can also be prepared by gene manipulation by methods known to the skilled worker. They include not only the naturally occurring growth factors but also derivatives or variants having substantially the same biological activity.

Thus, for example, TPO can be purchased commercially from CellSystems GmbH, St Katharinen. The use of human TPO is preferred for cultivating human adult hepatocytes. In addition, the preparation and characterization of TPO and its variants is described for example in EP 1 201 246, WO 95/21919, WO 95/21920 and WO 95/26746.

Suitable TPO variants are the TPO derivatives described in WO 95/21919 or the allelic variants or species homology described in WO 95/21920 or the pegylated TPO described in WO 95/26746 and EP 1 201 246, without restriction thereto. Pegylated TPO means for the purposes of the present invention TPO derivatives which are linked to an organic polymer such as, for example, polyethylene glycol, polypropylene glycol or polyoxyalkylene. Further variants of TPO also mean derivatives of TPO which have a sequence identity of less than 100% and nevertheless have the activity of TPO, as described preferably in EP 1 201 246. TPO derivatives normally have a sequence identity of at least 70%, preferably at least 75%, especially at least 80% and in particular at least 85% compared with human TPO including fragment a thereof having TPO activity. A particularly preferred TPO activity for the purposes of the present invention is the speeding up of proliferation, differentiation and/or maturation of megakaryocytes or megakaryocyte precursors in platelet-producing forms of these cells by TPO or its variants.

EPO is also referred to as embryonic form of TPO and is described with its variants for example in EP 0 148 605, EP 0 205 564, EP 0 209 539, EP 0 267 678 or EP 0 411 678.

The examples of derivatives and variants described in detail above apply analogously also to the other growth factors mentioned.

The term growth factor is accordingly not restricted according to the present invention to the naturally occurring forms, but also includes non-naturally occurring forms and variants or derivatives. The term growth factor includes according to the present invention not only growth promoters but also growth inhibitors such as, for example, somatostatin, TGF beta and/or prostaglandins. Such growth inhibitors are particularly suitable for suppressing or inhibiting the growth of mutated cells such as, for example, tumor cells, by highly concentrated local use thereof simultaneously or sequentially, for example also by means of hydrogels or slow-release materials.

The growth process of the invention is carried out in a culture suitable for the particular cells. It is possible in this connection by means of a suitable device for the cell aggregates formed where appropriate during the growth process to be broken up and, where appropriate, encapsulated and, where appropriate, frozen.

An example of a suitable device is a grid having, for example, a cutting mesh structure for example 500 μm in size, which has the effect that new subsidiary aggregates of, for example, hepatocytes can be repeatedly produced. This can advantageously take place in a completely closed system. It is possible in particular to employ contactless, automatically or manually controlled pumping systems which consist for example of piston pumps or generate directed flows generated magnetically or by compressed air compression of tubings. In the presence of endothelial cells it is possible through the shear stress in a perfused bioreactor for spontaneous confluence of the endothelial cells on the surfaces of the aggregates to occur, which may be advantageous for further use.

Materials suitable for the encapsulation are suitable ones which are known to the skilled worker and in which, for example, structured shapes or spaces are integrated and make an in situ growth structure or enlargement possible. An alternative possibility is for the capsule to be dispensed with and, for example, an endothelialization and thus optimal hemocompatibility to be achieved in the presence of endothelial cells.

In a further embodiment, the growth process of the cells is locally initiated and terminated, and structurally guided, preferably by a biological matrix.

The biological matrix is in this case for example treated with one of said growth factors or with a combination of said growth factors as mixture or sequentially. This makes 3-D regeneration and/or artificial guidance of tissue repair or tissue culturing possible even with adult cell systems.

The biological matrix is normally an implant, e.g. a stent, a patch or a catheter, a transplant, e.g. a skin transplant and/or a supporting material for the growth of cells, e.g. a so-called slow release material, e.g. a hydrogel for example based on fibrin and/or polymers such as, for example, polylactide or polyhydroxy-alkanoate, and/or alginates, a bone substitute material, e.g. tricalcium phosphate, an allogeneic, autologous or xeinogeneic acellularized or non-acellularized tissue, e.g. a heart valve, venous valve, arterial valve, skin, vessel, aorta, tendon, comea, cartilage, bones, tracea, nerve, miniscus, intervertebral disc, ureters, urethra or bladder (see, for example, EP 0 989 867 or EP 1 172 120), a matrix such as, for example, a laminin, collagen IV and/or Matrigel matrix, preferably a feeder layer such as, for example, collagen I, 3T3 and/or MRC-5 feeder layer, or a collagen fabric.

In a further preferred embodiment, the biological matrix is precolonized with cells, preferably tissue-specific cells, precursor cells, bone marrow cells, peripheral blood, adipose tissue and/or fibrous tissue, e.g. with adult precursor cells from the bone marrow, by methods known to the skilled worker. It is possible in this way to achieve anticipation of the in vivo wound-healing process in vitro, and thus a shortened reintegration time takes place after implantation in vivo.

The cells used according to the present invention are in particular adult cells, i.e. primarily differentiated cells which preferably no longer have an embryonic or fetal phenotype, particularly preferably human adult cells. Examples thereof are adult progenitor cells, tissue-specific cells, preferably osteoblasts, fibroblasts, hepatocytes and/or smooth muscle cells.

However, it is also possible to suppress or inhibit mutated cells such as, for example, tumor cells, by for example highly concentrated, simultaneous or sequential dosage of growth inhibitors such as somatostatin, TGF beta and/or prostaglandins. It is possible in this case to employ the hydrogels or slow-release materials which have already been mentioned and which comprise at least one of said growth inhibitors or are supplemented therewith, and are applied locally or in the vicinity of the mutated cells.

The method of the invention is thus particularly suitable for locally specific and/or directed multiplication, structural growth and subsequent differentiation of adult cells and/or for the regeneration of bones, tissues and/or endocrine organs, e.g. of heart valves, venous valves, arterial valves, skin, vessels, aortas, tendons, comea, cartilage, bones, traces, nerves, miniscus, intervertebral disc, ureters, urethra or bladders.

The method of the invention can also be employed for local administration in vivo by said growth factors being employed either alone or in combination as mixture or sequentially, or in combination with said biological matrices or supporting structures, for example for tissue regeneration, such as, for example, liver regeneration, myocardical regeneration or for wound healing in the region of the skin, e.g. for diabetic ulcers, or gingiva. For example, it is possible for, for example, TPO to be applied in a hydrogel, e.g. fibrin and/or a polymer such as, for example, polylactide or polyhydroxyalkanoate, and/or an alginate, to the resection surface for example of a liver for liver regeneration, or to be administered locally or systemically in, for example, acute liver failure via a port with the aid of a catheter. Said growth factors can thus be administered for example before, during or after a liver resection or removal of liver tissue in order to assist liver regeneration. On use of said growth factors for promoting cartilage regeneration, the growth factor(s) can be injected directly into the knee joint. It is thus possible for the growth factor(s) to act via the sinovial fluid directly on the formation of a new cartilage structure.

Consequently, the present invention also relates to the use of the growth factors TPO and/or EPO and/or GH and/or somatostatin and/or LIF and/or CNTF for producing a medicament for the treatment of regeneration of bones, cartilage, tissues and/or endocrine organs, e.g. parenchymal and/or non-parenchymal organs, especially of myocardium, heart valves, venous valves, arterial valves, skin, vessels, aortas, tendons, comea, cartilage, bones, tracea, nerves, miniscus, intervertebral disc, liver, intestinal epithelium, ureters, urethra or bladders, or for the treatment of degenerative disorders and/or for assisting the wound healing process, especially in Crohn's disease, ulcerative colitis and/or in the region of the skin, preferably for diabetic ulcers or gingiva and/or for the treatment of liver disorders, especially of cirrhosis of the liver, hepatitis, acute or chronic liver failure and/or wound healing in the muscle region after sports injuries, muscle disorders, bone injuries, soft tissue injuries and/or for improving wound healing and tissue regeneration, e.g. after operations, acute and chronic disorders and/or for improving wound healing and tissue regeneration, for example after operations, acute and chronic disorders and/or ischemic myocardial disorders for stimulating neoangiogenesis and regeneration and/or ischemias after injuries and trauma and/or regeneration of tissues following a tissue injury, e.g. with myocardial infarction or thromboses (central or peripheral) in some circumstances with subsequent ischemia. EPO dosage in this case makes neoangiogenesis and subsequent or accompanying tissue regeneration possible.

In a particular embodiment there is use as growth factor in addition of transforming growth factor beta (TGF beta), prostaglandins, granulocyte-macrophage stimulating factor (GM-CSF), growth hormone releasing hormone (GHRH), thyrotropin-releasing hormone (TRH), gonadotropin-releasing hormone (GnRH), corticotrophin-releasing hormone, dopamine, antidiuretic hormone (ADH), oxytocin, prolactin, adrenocorticotropin, beta-celltropin, lutrotropin and/or vasopressin, or additionally of one or more nerve regeneration factors, preferably nerve growth factor (NGF) and/or one or more vessel regeneration factors, preferably vascular endothelial growth factor (VEGF) and/or platelet derived growth factor (PDGF).

The further embodiments described in the present invention apply analogously also to the described uses of the invention.

A further possibility is for a biological matrix or supporting structure comprising at least one of the growth factors TPO, EPO, GH, especially HGH, somatostatin, LIF and/or CNTF, to be used as inductive substrate for 3-D growth and/or regeneration within a multiplication phase or after a multiplication phase for differentiation or for growth arrest. For example, at least one of said growth factors can be applied to a stent in combination with a so-called slow-release material, as described by way of example above.

The present invention therefore relates further also to a biological matrix or supporting structure comprising at least one of the growth factors thrombopoietin (TPO), erythropoietin (EPO), growth factor (GH), especially human growth hormone (HGH), somatostatin, leukemia inhibitory factor (LIF) and/or ciliary neurotropic factor (CNTF), where the biological matrix or supporting structure in this may also additionally comprise at least one of the growth factors TGF beta, prostaglandin, GM-CSF, GHRH, TRH, GnRH, CRH, dopamine, ADH, oxytocin, prolactin, adrenocorticotropin, beta-celltropin, lutrotropin and/or vasopressin and, where appropriate, additionally one or more nerve regeneration factors, preferably nerve growth factor (NGF) and/or one or more vessel regeneration factors, preferably vascular endothelial growth factor (VEGF) and/or platelet derived growth factor (PDGF).

The biological matrix or supporting structure of the invention is, for example, an implant, a transplant and/or a supporting material for the growth of cells, the biological matrix or supporting structure possibly being a stent, a catheter, a skin, a hydrogel, a bone substitute material, an allogeneic, autologous or xenogeneic, acellularized or non-acellularized tissue, a synthetic tissue, a feeder layer or a fabric such as, for example, a fabric made of collagen, laminin and/or fibronectin with or without synthetic or other type of basic structure, such as, for example, plastic or a biological matrix. Exemplary embodiments have already been described above.

The biological matrix or supporting structure is, as already described above in detail, preferably already precolonized with tissue-specific cells, precursor cells, bone marrow cells, peripheral blood, adipose tissue and/or fibrous tissue, or already prepared for in vivo colonization or inductive remodeling in vitro.

The biological matrix or supporting structure can also be coated with a (bio)polymer layer which comprises at least one of said growth factors. Fibrin, plasma, collagen and/or polylactides are suitable for example as (bio)polymer layer.

The present invention also relates to a method for producing a biological matrix or supporting structure of the invention, in which an optionally activated biological matrix or supporting structure is coated with at least one of the growth factors TPO, EPO, GH, in particular HGH, somatostatin, LIF and/or CNTF, where said matrix or supporting structure can optionally be coated with additionally at least one of the growth factors TGF beta, prostaglandin, GM-CSF, GHRH, TRH, GnRH, CRH, dopamine, ADH, oxytocin, prolactin, adrenocorticotropin, beta-celltropin, lutrotropin and/or vasopressin and, where appropriate, additionally with one or more nerve regeneration factors, preferably NGF and/or one or more vessel regeneration factors, preferably VEGF and/or PDGF.

The activation of the biological matrix or supporting structure can take place for example by means of plasma ionization, e.g. using hydrogen peroxide, or by means of laser activation.

An alternative possibility is a coating with a biodegradable (bio)polymer layer which comprises said growth factor(s). Suitable examples for this purpose are fibrin, plasma, blood, collagen and/or polylactides.

It is likewise possible in the method of the invention for the biological matrix or supporting structure to be precolonized in vitro with cells, preferably tissue-specific cells, precursor cells, bone marrow cells, peripheral blood, adipose tissue and/or fibrous tissue.

The preferred features or feature examples of the present invention which are described above apply analogously to the production process of the invention.

The present invention also extends to a device for carrying out the method of the invention, where a perfused bioreactor, especially in the form of a closed system, is preferred.

The following examples are intended to explain the invention in detail without restricting it.

### EXAMPLES

1. Bone Regeneration

A single-phase beta tricalcium phosphate is prepared as granules with a microporosity of, for example, >15 μm and shaped in a mold of a 3-D defect corresponding to a patient's requirement. This normally takes place in a sintering process. The material is subsequently treated by plasma ionization so that activation of the surfaces occurs and the construct is placed in a solution with thrombopoietin, erythropoietin and/or growth hormone (GH) and thus coated in small quantities in a defined way. Alternatively, as incubation in a solution without previous surface activation or a coating with a biodegradable (bio)polymer layer comprising these growth factors can take place. It is possible in this case to employ for example fibrin, plasma, collagen and/or polyactides.

This construct is then either immediately introduced into a defect or precolonized in vitro with tissue-specific cells, precursor cells or bone marrow cells. This achieves anticipation of the in vivo wound-healing process in vitro and thus a shortened reintegration time can take place after implantation in vivo (e.g. after 7 days). A combination with factors of nerve regeneration (NGF) or vessel regeneration (VEGF, PDGF) is possible. Combination with the structure-forming factors and environment concepts is of interest in this connection.

In vivo and in vitro there is integration of the blood-forming and stem cell-rich bone marrow and an increased rate of differentiation of osteoblasts and an increased rate of absorption of the supporting matrix and a replacement by normal bone. Site-specific integration takes place owing to the recruitment competence and the inductive character.

This can be further promoted by colonization on the external sides with periosteum in vitro.

2. Heart Valve Regeneration and Production of Urological Constructs

A biological matrix (allogeneic or autologous heart valve with and without acellularization, a synthetic supporting structure made of plastics which resembles the physiological microenvironment of the cardiovascular target tissue in terms of the chemical composition of the collagens and their spatial arrangement) is precoated with thrombopoietin and erythropoietin as growth factors.

The material is then treated by plasma ionization (e.g. using hydrogen peroxide, H₂O₂), simultaneously achieving sterilization, so that activation of the surfaces occurs and the construct is placed in a solution with thrombopoietin, erythropoietin and/or growth hormone (GH) and thus coated in small quantities in a defined way. Alternatively, incubation in a solution without previous surface activation or coating with a biodegradable (bio)polymer layer comprising these growth factors is possible. Fibrin, plasma, blood, collagen or polyactides can be employed in this case.

This construct is then either immediately introduced at the required site (heart valve position, as patch or vessel replacement) or precolonized in vitro with tissue-specific cells, precursor cells or bone marrow cells. This achieves anticipation of the in vivo wound-healing process in vitro and thus a shortened reintegration time can take place after implantation in vivo (e.g. after 7 days). A combination with factors of nerve regeneration (NGF) or vessel regeneration (VEGF, PDGF) is possible, but not absolutely necessary.

In vivo and in vitro there is integration of the blood-forming and stem cell-rich bone marrow and an increased rate of differentiation of fibroblasts and smooth muscle cells and an increased rate of absorption of the supporting matrix and a replacement by normal cardiovascular tissue. Site-specific integration takes place owing to the recruitment competence and the inductive character.

This can be further promoted by colonization on the external sides with endothelial cells in vitro.

Urological constructs can be produced in a corresponding manner.

3. Multiplication of Adult Hepatocytes in Coculture with Nonparenchymal Cells

A mixed liver cell population from a biopsy or a partial sectate are treated with TPO and/or EPO and/or growth hormone, e.g. HGH in a concentration of 10-50 ng/ml by addition to the medium supernatant. The seeding cell density is 10 000 cells/cm. After confluence is reached, the cells are treated with 0.005% collagenase and 0.01% trypsin with the addition of 2 g/l albumin or autologous serum (10-20%) for 5 h. The cells are then aspirated off and washed three times in culture medium (Williams E (Williams et al. (1971) Exptl. Cell Res., 69, 106) with 2 g/l albumin and then put for sedimentation in a collagen-coated Petri dish.

Differentiation of the cells can be achieved by overlayering with an extracellular matrix.

Alternatively, the cells can be prevented from sedimenting by agitation and come together for the aggregation.

In order to avoid too great an enlargement of the aggregates during the growth process, the cells can be passed in an appropriate device over a grid having a cutting mesh structure 500 μm in size, so that new subsidiary aggregates can be repeatedly produced. This can take place in a completely closed system. Ideally, contactless pumping systems (no squeezing by peristaltic systems but directed flows generated magnetically or by compressed air compression of tubings, or piston pumps—automatic or manual) are employed.

The cells can than be encapsulated and frozen. Structured shapes and spaces can be integrated in the capsule structure, which makes an in situ growth structure and enlargement possible.

Alternatively, the capsule can be dispensed with and, through the presence of the endothelial cells in this system and targeted addition of these cells, an endothelialization and thus optimal hemocompatibility can be achieved.

The shear stress in a perfused bioreactor results in spontaneous confluence of the endothelial cells on the surfaces of the aggregates. When the target size is reached, they can be frozen for example in the bags which are already ideally used for the culture.

4. Soft Tissues (Muscle Patches, Nerves, Tendons)

For reconstructing abdominal wall defects it is possible to produce collagen tile or fabrics such as laminin, fibronectin with or without synthetic or another type of basic structure such as, for example, plastic or a biological matrix, or spatially defined structures (tubes for nerves, tendons) correspondingly as above. These collagen tile or structures are shaped, coated with TPO, EPO and/or growth hormone (GH) and implanted or precolonized with cells of the target tissue (e.g. tenocytes, neurons).

A biological matrix (allogeneic or autologous heart valve with and without acellularization, a synthetic supporting structure made of plastics which resembles the physiological microenvironment of the target tissue in terms of the chemical composition of the collagens and their spatial arrangement) is precoated with thrombopoietin and erythropoietin as growth factors.

Subsequent or prior to this the material is then treated by plasma ionization (e.g. using hydrogen peroxide, H₂O₂), simultaneously achieving sterilization, so that activation of the surfaces occurs and the construct is placed in a solution with thrombopoietin, erythropoietin and/or growth hormone and thus coated in small quantities in a defined way. Alternatively, incubation in a solution without previous surface activation or coating with a biodegradable (bio)polymer layer comprising these growth factors is possible. Fibrin, plasma, collagen and/or polylactides can be employed in this case.

This construct is then either immediately introduced at the required site (abdominal wall, myocardium, skeletal muscle as patch) or precolonized in vitro with tissue-specific cells, precursor cells or bone marrow cells. This achieves anticipation of the in vivo wound-healing process in vitro and thus a shortened reintegration time can take place after implantation in vivo (e.g. after 7 days). A combination with factors of nerve regeneration (NGF) or vessel regeneration (VEGF, PDGF) is possible, tout not absolutely necessary.

In vivo and in vitro there is integration of the blood-forming and stem cell-rich bone marrow and an increased rate of differentiation of fibroblasts and smooth muscle cells and an increased rate of absorption of the supporting matrix and a replacement by normal cardiovascular tissue. Site-specific integration takes place owing to the recruitment competence and the inductive character.

This can be further promoted by in vitro colonization on the external sides with keratinocysts (abdominal muscle), Schwann's cells and/or fibrous tissue.

5. Regeneration of Tissues in Vivo

a) Liver

After partial resection of the liver, EPO is administered systemically and/or topically to the patient by application to the resection surface in conjunction with a polymer. The polymer may be a biopolymer such as, for example, fibrin (from, for example, fibrin glue), polymerized plasma, polymerized blood or bioadhesives, e.g. mussel adhesive. However, it may also be synthetic or biological gels or hydrogels. The EPO can also be introduced into fabrics which serve to stop bleeding (e.g. collagen fabrics, tamponade, wovens and knits).

Through the action of EPO there is restoration of the original volume of the liver within 2 weeks. This involves not only a multiplication of the hepatocytes but also a coordinated growth in which the vessels, the bile ducts and the capsular structures also grow back to their original size.

It was possible to show in 30 animals that regeneration of the liver took place significantly compared with the control animals (without EPO dosage).

EPO can also be employed for regenerating the liver in chronic liver disorders such as, for example, cirrhosis, fibrosis, hepatitis. It is thus possible for the first time to achieve a therapeutic effect in relation to the liver parenchyma.

b) Inflammatory Bowl Disorders

In patients with Crohn's disease, wound healing in the region of the intestinal epithelium is impaired. Underlying tissue structures may also be involved in inflammatory reactions. In these patients, systemic and/or topical dosage of EPO leads to restoration of the intestinal epithelium through regeneration. Topical dosage may take place by slow release capsules in the intestinal region or by giving suppositories with gels or local installation with solutions.

Absorption in the regional vascular area can be optimized by giving pegylated (PEG) compounds, so that systemic effect and thus initiation of the wound-healing process can take place via the regional dosage in the area of inflammation.

The presences of an anemia is to be regarded as a prognostic positive factor for patients with Crohn's disease. It was assumed in the past that the anemia is an independent concomitant disorder or is attributable to the wasting due to absorption problems. Our results show that the impairment of wound healing involves a deficiency of endogenous EPO. It is thus possible to treat Crohn's disease very selectively by exogenous dosage of EPO. Further uses are to be found also in the area of ulcerative colitis.

c) Impairments of Wound Healing in the Region of the Skin

Patients with diabetic ulcers have trophic disorders which make wound closure in the region usually of the legs difficult. The capacity for structural tissue regeneration is restricted owing to the basic disorder. In these cases, wound healing is induced through systemic and/or topical dosage of EPO. It proves to be advantageous to administer EPO after roughening of the lower stratum during a debridement. The combination of EPO with a polymerization induced by calcium chloride leads to integration of EPO in a blood clot, resulting in a topical slow-release preparation. Alternatively, EPO can also be administered in conjunction with a fibrin glue or with a fabric or with a tamponade (e.g. collagen fabric) impregnated with EPO.

EPO can be given in a similar manner for all other wound healing requirements, e.g. in the muscle region after sports injuries, muscle disorders, bone injuries, soft-tissue injuries and generally for improving wound healing and tissue regeneration, e.g. after operations, acute and chronic disorders.

1-27. (canceled)

28. A method for the topical treatment of epithelium during the healing of a wound comprising applying to the wound a combination of erythropoietin (EPO) and a biological matrix selected from the group consisting of a hydrogel, a synthetic tissue, a patch, a fabric, a fibrin glue a tamponade or a feeder layer.

29. The method of claim 28, wherein the epithelium is gingivia.

30. The method of claim 28, wherein the epithelium is skin.

31. The method of claim 28, wherein the wound is a diabetic ulcer.

32. The method of claim 28, wherein the wound is liver resection.

33. The method of claim 28, wherein the combination further comprises calcium chloride suitable for clotting blood after roughening of a lower stratum of the epithelium during debridement,

34. A combination of erythropoietin (EPO) and a biological matrix selected from the group consisting of a hydrogel, a synthetic tissue, a patch, a fabric, a fibrin glue, a tamponade or a feeder layer for use for the manufacture of a medicament for the topical treatment of an epithelium during the healing of a wound.
